(19) United States
(12) Patent Application Publication
(10) Pub. No.: US 2022/0015429 A1
(43) Pub. Date: Jan. 20, 2022

(54) Heating Chamber Lid

(71) Applicant: JT International S.A., Geneva (CH)

(72) Inventors: Nicholas Alan Brown, Deepcut (GB); Nicholas Hedley Chubb, London (GB)

(73) Assignee: JT International S.A., Geneva (CH)

(21) Appl. No.: 17/278,790

(22) PCT Filed: Nov. 15, 2019
(86) PCT No.: PCT/EP2019/081494

§ 371 Date: Mar. 23, 2021

(30) Foreign Application Priority Data
Nov. 16, 2018 (EP) 18206781.9

(51) Int. Cl. A24F 40/46; A24F 40/42; A24F 40/57

(52) U.S. Cl. CPC A24F 40/46; A24F 40/57; A24F 40/42

(57) ABSTRACT

A vapour generating device includes a heating chamber arranged to receive a vapour generating substance and a lid arranged to close the heating chamber. The device further includes a locking mechanism arranged to prevent the lid from moving from a closed position to an open position when a temperature of the heating chamber exceeds a predetermined threshold. The locking mechanism has a portion which changes its shape and/or volume based on its temperature and the change activates the locking mechanism.

### FIELD OF THE INVENTION

The present invention relates to a lid for a heating chamber in a vapour generating device.

### BACKGROUND

Devices which heat, rather than burn, a substance to produce a vapour for inhalation have become popular with consumers in recent years.

Many hand-held vapour generating devices comprise a heating chamber, or oven, connected to an outlet. A vapour generating substance is placed inside the chamber and the heating mechanism causes a vapour to be generated, which can be inhaled by the user through the outlet such as mouthpiece.

Many hand-held vapour generating devices require the user to access the heating chamber and insert the vapour generating substance into the heating chamber. Requiring the user to access internal parts of the device may lead to injury, for example due to the heating chamber still being hot.

Furthermore, if the structure is complex, the structures increase the likelihood that the user does not correctly close the heating chamber before using the vapour generating product, which may result in the user coming into contact with parts of the heating chamber that may still be hot.

It would be desirable to provide a vapour generating device which provides improved safety whilst keeping the structure of the device simple to use. The present invention therefore seeks to overcome at least some of the above problems.

### SUMMARY OF THE INVENTION

According to an aspect of the present invention there is provided a vapour generating device comprising a heating chamber arranged to receive a vapour generating substance, a lid arranged to close the heating chamber, and a locking mechanism arranged to prevent the lid from moving from a closed position to an open position when a temperature of the heating chamber exceeds a predetermined threshold, wherein the locking mechanism has a portion which changes its shape and/or volume based on its temperature and the change activates the locking mechanism. The vapour generating device is a hand-held vapour generating device. The vapour generating device further comprises a vapour-generating unit arranged to generate a vapour from the vapour-generating substance and a mouthpiece, in fluid communication with the heating chamber, for allowing a user to inhale the generated vapour.

Advantageously, this ensures that the behaviour of the locking mechanism is dependent on the temperature of the locking mechanism. It is therefore not possible for a user to override the locking mechanism if the temperature of the heating chamber is considered unsafe. As the locking mechanism is directly correlated with the temperature of the heating chamber the user is protected from burning.

The device therefore provides a simple structure which prevents the user from accidentally coming into contact with the heating chamber. This ensures that a user is only able to open the lid of the heating chamber and access the inside of the heating chamber, for example to insert vapour generating substance or to clean the chamber, when the temperature of the heating chamber has reached a safe value. This ensures that the user is protected from burning as the locking mechanism is directly related to the temperature of the heating chamber.

The locking mechanism may comprise a bi-metallic portion. That is, the portion of the locking mechanism which changes its shape and/or volume based on its temperature may comprise a bi-metallic strip. The bi-metallic portion may be a bi-metallic strip. This may provide a simple mechanism of converting a temperature change into a mechanical displacement, which can be used to lock the lid of the heating chamber.

The vapour generating device may comprise a biasing mechanism arranged to bias the lid in an open position. The lid is therefore easy to open, ensuring simple functionality for the user.

The locking mechanism may comprise a locking member arranged to retain the lid in the closed position. The locking member may be a locking pin. Advantageously, the lid of the heating chamber is prevented from inadvertently opening. This ensures that the user does not accidentally come into contact with a hot heating chamber and reduces the likelihood of injury to the user.

The locking member may be moveable between a first position and a second position. The locking member may be arranged to prevent the lid from moving from a closed position to an open position when the locking member is in the first position. The locking member may be arranged to allow the lid to move from a closed position to an open position when the locking member is in the second position. Two distinct positions for the locking mechanism ensures that the locking mechanism cannot accidentally become unlocked, preventing the user from becoming injured.

The locking member may be biased towards the first position. Advantageously, the lid of the heating chamber is prevented from inadvertently opening. This ensures that the user does not accidentally come into contact with a hot heating chamber and reduces the likelihood of injury to the user.

The locking mechanism comprises at least one intermediate part arranged to be connected to the bi-metallic portion of the locking mechanism. The at least one intermediate part may comprise a rod, plate, or block. This provides a flexible design for the locking mechanism whilst keeping the design and structure simple.

The at least one intermediate part may be moveable between a third position and a fourth position. The third position may correspond to the locking mechanism being in a locked position and the fourth position may correspond to the locking mechanism being in an unlocked position. When the temperature of the heating chamber exceeds a predetermined threshold, the bi-metallic portion may be arranged to have a shape such that the intermediate part is in the third position. When the temperature of the heating chamber is lower than a predetermined threshold, the bi-metallic portion may be arranged to have a shape such that the intermediate part is in the fourth position. This provides a simple and reliable structure for the locking mechanism.

The locking member may be arranged to be in the first position when the intermediate parts are in the third position. When in the second position, the locking member is prevented from moving to the second position. This provides a simple a reliable structure for the locking mechanism.

When the temperature of the heating chamber exceeds the predetermined threshold, the bi-metallic portion may be arranged to move the intermediate part into the third position. Advantageously, this provides a simple and reliable structure for the locking mechanism as the locking mechanism does not rely on the interaction between multiple parts. Instead, the locking mechanism is activated and deactivated by the temperature of the heating chamber and the corresponding movement of the bi-metallic portion. A lack of mechanical parts reduces the likelihood of the locking mechanism failing ensuring the device is safe to use.

The locking member may comprise an outer surface which is exposed. The locking member may be arranged to be moved from the first position to the second position by a force applied to the outer surface. Having an exposed outer surface on the locking member ensures that the user is easily able to locate and interact with the locking member to open the lid. No additional parts are required in order for the user to unlock the lid, helping keep the device simple and easy to use. The locking member may be in the form of a release button. The release button may allow the user to easily open and operate the lid, especially if the lid is biased towards the open position. The easy-open functionality is a result of the fact that all the user needs to do to open the lid is to push the locking member.

The lid may comprise an engaging portion which may be arranged to releasably engage with the locking member. The engaging portion may comprise a hook-like structure. The hook-like structure may be arranged to releasably engage with a complimentary hook-like structure on the locking member. This may ensure that the lid does not accidentally become unlocked unless the user has initiated the unlocking mechanism. Advantageously, the chance of injury to the user is reduced.

The lid may comprise any suitable mechanism for allowing the lid to open and close, for example a hinge mechanism or a sliding mechanism. In some embodiments the mechanism is a sprung hinge. These simple mechanisms ensure the lid is easy to open by the user, facilitating use of the device.

The bi-metallic portion may comprise any suitable shape which allows for a locking and an unlocking configuration for example, a plate shape or a rod shape. These shapes are simple to manufacture and incorporate into the vapour generating device keeping the construction of the device simple. Preferably the bi-metallic portion, such as a plate shape or a rod shape, has longitudinal direction wherein one end of the longitudinal direction is fixed to the device and another end of the longitudinal direction is fixed to or contacts the intermediate part, at least when the bi-metallic portion is heated. This arrangement helps ensure correct movement of the intermediate part.

The vapour generating device may comprise a heater arranged to heat the heating chamber. The vapour generating device may comprise a heat insulator. The heat insulator may be arranged between the bi-metallic portion and an outer surface of the vapour generating device. The outer surface of the vapour generating device is therefore prevented from becoming too hot allowing the user to pick up and hold the device, even when the heating chamber is too hot to open.

There may be no thermal insulation between the portion of the locking mechanism which changes its shape and/or volume based on its temperature and the heater. This ensures that more heat can travel to the portion of the locking member whose shape depends on the temperature. This portion is therefore more responsive to changes in temperature as the temperature of the portion more accurately reflects the temperature of the heating chamber which has been heated by the heater.

The vapour generating substance may be any suitable substance capable of forming a vapour. The substance may be solid or semi-solid substance. The substance may comprise plant derived material and in particular, the substance may comprise tobacco. Typically, the vapour generating substance is a solid or semi-solid tobacco substance. Example types of vapour generating solids include powder, granules, pellets, shreds, strands, porous material, foam, or sheets.

Preferably, the vapour generating substance may comprise an aerosol-former. Examples of aerosol-formers include polyhyrdric alcohols and mixtures thereof such as glycerine or propylene glycol. Typically, the vapour generating substance may comprise an aerosol-former content of between approximately 5% and approximately 50% on a dry weight basis. Preferably, the vapour generating substance may comprise an aerosol-former content of approximately 15% on a dry weight basis.

In some examples the vapour generating device may be able to detect the position of the lid. The vapour generating device may therefore comprise a sensor, which may be referred as a lid closing sensor. The sensor may be arranged to determine when the lid is in a closed or an open position. The sensor may comprise any suitable sensing mechanism for example the sensor may be a photo sensor or a mechanical sensor. The sensor may be arranged to send a signal to the heater to start heating the heating chamber only when the lid is determined to be in the closed position. This ensures that the heating chamber is not being heated when the lid is open which reduces the likelihood of a user injuring themselves on a hot heating chamber.

### BRIEF DESCRIPTION OF THE DRAWINGS

Embodiments of the present invention will now be described, by way of example only, with reference to the accompanying drawing in which:

FIG. 1 is a schematic view of a vapour generating device comprising a locking mechanism in an unlocked state;

FIG. 2 is a schematic view of a vapour generating device comprising a locking mechanism in a locked state; and

FIG. 3 is a schematic view of a vapour generating device comprising a sensor.

### SPECIFIC DESCRIPTION

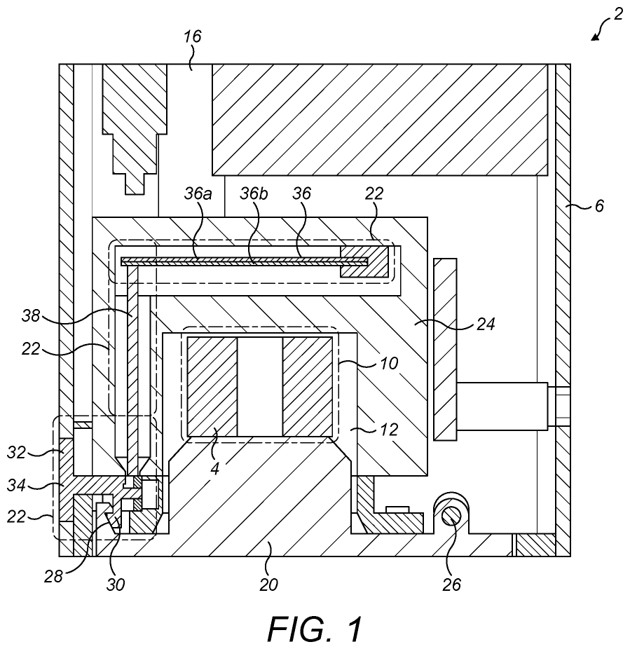

Part of a vapour generating device 2 is shown generally in FIG. 1. The vapour generating device 2 is a hand-held device, by which we mean that the device is able to be held and supported by a user un-aided in a single hand. The vapour generating device includes a heating chamber 4, which may also be referred to as an oven, for receiving solid vapour generating material, a vapour-generating unit 6 for generating a vapour from the vapour-generating substance, and a mouthpiece in fluid communication with the heating chamber 4. The vapour-generating unit 6 heats the vapour-generating substance inside the chamber 4, generating a vapour. The vapour is then able to be inhaled by a user at the mouthpiece. The vapour-generating substance may include at least one of tobacco, humectant, glycerine, and propylene glycol. However, any other volatile substance could be used from which a vapour or aerosol can be generated.

The user inhales the vapour by drawing air into the device 2, through the chamber 4 containing the vapour-generating substance, and out of the mouthpiece when the chamber 4 is heated. Heating is achieved by the chamber 4 being located in a heating portion 10 of the vapour-generating unit 6 surrounded by a heating element, or heater. The chamber 4 is in fluid communication, via an airflow passageway 16, with an air inlet formed in the vapour-generating device 2 and an air outlet in the mouthpiece when the device 2 is assembled. Air is drawn through the device by application of negative pressure, which is usually created by a user drawing air from the air outlet.

The vapour generating device further includes a lid 20 for retaining the vapour generating material inside the heating chamber 4 and a locking mechanism 22 for retaining the lid 20 in a closed position. The lid 20 allows the heating chamber 4 to be opened and closed so the user can access the inside of the heating chamber 4 while the locking mechanism 22 prevents the user from opening the lid 20 when the temperature of the heating chamber 4 exceeds a certain temperature, as will be explained in more detail.

The structure of the heating chamber 4 and locking mechanism 22 can be more clearly seen in FIG. 1. The heating portion 10 and heating chamber 4 are substantially surrounded by a heat insulator 24, in the form of insulation 24. The insulation 24 does not surround an opening of the heating chamber 4, which is sealed by the lid 20, to ensure that the user is able to access the heating chamber 4 for the insertion of vapour generating substance. In some examples, the lid 20 may also include insulation.

The lid 20 is pivotally attached to the vapour generating device 2 via a sprung hinge 26. The sprung hinge 26 is arranged to bias the lid 20 in the open position. The presence of the bias in the sprung hinge 26 allows the lid 20 to behave as a quick release lid 20 in that once the user desires to open the lid 20 of the heating chamber 4, the lid 20 will quickly move from its closed position to its open position.

The locking mechanism 22 is provided in order to retain the lid 20 in its locked position, against the bias of the sprung hinge 26. The locking mechanism 22 comprises two parts, a first part 28 and a second part 30, which are arranged to releasably engage with each other. The first and second parts 28, 30 of the locking mechanism 22 are formed as complimentary hook-like structures. The first part 28, or first hook structure 28, is formed as part of the lid 20, opposite the sprung hinge 26. The second part 30, or second hook structure 30, is formed as part of a release button 32. The release button 32 is activated by the user to disengage the locking mechanism 22, allowing the lid 20 of the vapour generating device 2 to open so the user can access the heating chamber 4.

The release button 32 is positioned on the side of the vapour generating device 2 so that a surface 34 of the release button 32 is outwardly facing, forming part of the external surface of the vapour generating device 2. To activate the release button 32, and open the lid 20, the user presses on the outer surface 34 of the button 32, towards the main body of the vapour generating device 2. The pressing action moves the release button 32 inwards towards the main body of the vapour generating device 2. As the second hook structure 30 of the locking mechanism 22 is part of the release button 32, the lateral inward movement of the release button 32 causes lateral inward movement of the second hook structure 30. This lateral movement causes the second hook structure 30 to disengage from the first hook structure 28, which remains stationary. Once the two hook structures 28, 30 are disengaged the sprung hinge 26 causes to lid 20 to spring open.

In order to prevent the user from opening the lid 20 of the vapour generating device 2 when the heating chamber 4 is still hot, the locking mechanism 22 is a safety locking mechanism. By this we mean that the locking mechanism 22 further includes a safety device which prevents the user from unlocking the locking mechanism 22 and opening the lid 20 when the temperature inside the heating chamber 4 is considered too high to be safe.

The safety device of the locking mechanism 22 comprises bi-metallic parts 36 in the form of a bi-metallic strip 36. The bi-metallic strip 36 is located above the heating chamber 4 within the body of the vapour generating device 2, as can be seen in FIG. 1. The bi-metallic strip 36 is also surrounded by the insulation 24 which substantially surrounds the heating chamber 4.

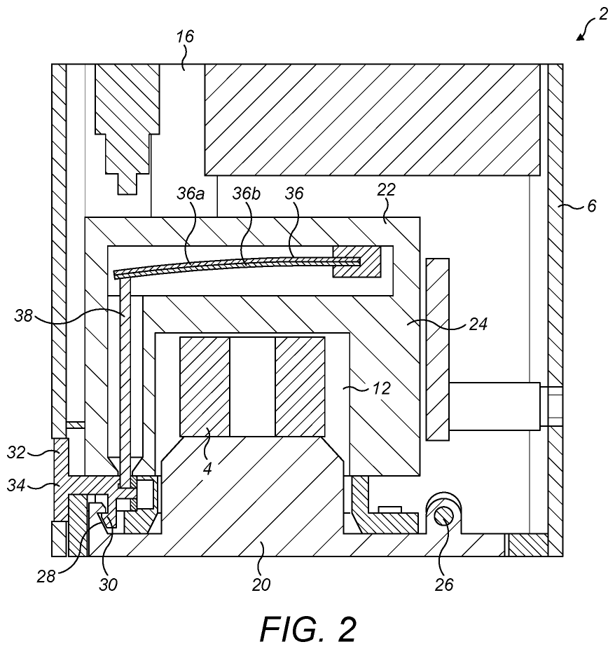

The bi-metallic strip 36 comprises two strips 36a, 36b of different metals which exhibit different thermal expansion rates when they are heated. The two metal strips 36a, 36b are joined together along their lengths using standard techniques such as welding or brazing. As a result of the different expansion rates, the bi-metallic strip 36 bends away from its longitudinal axis in one direction when it is heated and in the opposite direction when it is cooled. For example, when the bi-metallic strip 36 of the vapour generating device 2 is heated, it will bend outwardly, away from the main body of the vapour generating device 2 towards the heating chamber 4, as shown in FIG. 2. When the bi-metallic strip 36 of the vapour generating device 2 cools down, it will bend inwardly, towards the main body of the vapour generating device 2 away from the heating chamber 4 and will return to its original horizontal position, as shown in FIG. 1.

The temperature at which the bi-metallic strip 36 bends is determined by the two metals of which the bi-metallic strip 36 is made from. The two metals are chosen so that when the temperature of the heating chamber 4 exceeds a particular threshold temperature, the bi-metallic strip 36 will heat up and bend towards the heating chamber 4. Once the temperature of the heating chamber 4 has fallen below the threshold temperature, the bi-metallic strip 36 will cool down and return to its original configuration. The threshold temperature is a temperature above which it would be unsafe for a user to come into contact with the heating chamber 4, or any other parts of the vapour generating device 2 which heat up during usage, and pose a risk of burning.

The locking mechanism 22 further comprises an intermediate part 38 of the locking mechanism, which may be in the form of a rod as shown in FIG. 1. The intermediate part is vertically moveable between a locking position illustrated in FIG. 2, in which the locking mechanism 22 is considered locked, and an unlocking position as illustrated in FIG. 1, in which the locking mechanism 22 is considered unlocked. The intermediate part 38 is located so that it can come into contact with the bi-metallic strip 36 and the second hook structure 30.

The movement of the bi-metallic strip 36 through the change in its shape operates the intermediate part and causes it to move between the locking and unlocking positions. In particular, when the bi-metallic strip 36 bends downwards towards the heating chamber 4 it comes into contact with the intermediate part 38 and forces the intermediate part 38 downwards, as shown in FIG. 2. The downward movement of the intermediate part 38 causes it to come into contact with, and interrupt, the second hook structure 30 as shown in FIG. 1. The intermediate part 38 therefore prevents lateral movement of the second hook structure 30 inwardly towards the main body of the vapour generating device 2. This means that a user is not able to press on the release button 32 to unlock the lid 20 of the heating chamber 4 when the intermediate part 38 has moved downwards into its locking position.

As the heating temperature cools 4 down the bi-metallic strip 36 will also cool down and its shape will return to its original flat, horizontal shape. As the bi-metallic strip 36 cools it will exert less force on the intermediate part 38. Eventually, once the bi-metallic strip 36 has cooled completely, it will not exert any force on the intermediate part 38 and the intermediate part will move upwards into its unlocking position as shown in FIG. 1. The intermediate part 38 includes a biasing mechanism, biasing the intermediate part 38 into its unlocking position so that it returns to this position automatically once the bi-metallic strip 36, and heating chamber 4, have cooled down to a suitable temperature. Any suitable biasing mechanism can be used such as a spring mechanism.

The bi-metallic strip 36 therefore converts a temperature change into a mechanical displacement. This is important because the behaviour of the locking mechanism 22 is therefore dependent on the temperature of the locking mechanism 22. This ensures that it is not possible for a user to manually override the locking mechanism by interfering with the part of the locking mechanism 22. The dependency of the locking mechanism 22 on the temperature of the bi-metallic strip 36 forms the safety device of the safety locking mechanism. The user of the vapour generating device 2 is protected from burning themselves on the heating chamber 4 because the function of the locking mechanism 22 is directly related to the temperature of the heating chamber 4.

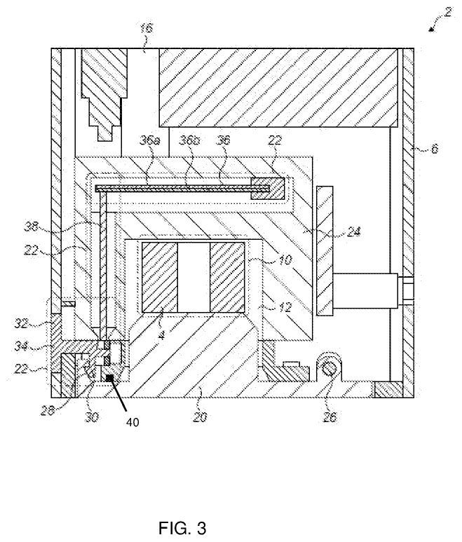

In some examples, the vapour generating device 2 includes a sensor 40, located near the locking mechanism 22, which is able to detect the position of the lid 20 as shown in FIG. 3. The sensor 40 is a photo sensor which is able to determine whether the lid 20 is in the closed position or the open position, depending on the amount of light which reaches the photo sensor 40. When the photo sensor 40 detects a large amount of light, the photo sensor 40 determines that there is nothing block the path of light to the sensor 40 and so it is determined that the lid 20 is in the open position. When the photo sensor 40 detects little or no light, the photo sensor 40 determines that there is an obstacle between the path of light and the photo sensor 40 and so it is determined that the lid 20 is in the closed position. The photo sensor 40 is arranged to send a signal to the heater to start heating the heating chamber only when the lid 20 is determined to be in the closed position. This ensures that the heating chamber is not being heated when the lid 20 is open which reduces the likelihood of a user injuring themselves on a hot heating chamber 4.

1. A hand-held vapour generating device comprising:
a heating chamber arranged to receive a vapour generating substance;
a vapour-generating unit arranged to generate a vapour from the vapour-generating substance;
a mouthpiece, in fluid communication with the heating chamber, for allowing a user to inhale the generated vapour;
a lid arranged to close the heating chamber; and
a locking mechanism arranged to prevent the lid from moving from a closed position to an open position when a temperature of the heating chamber exceeds a predetermined threshold, wherein the locking mechanism has a portion which changes its shape and/or volume based on its temperature and the change activates the locking mechanism.

2. The vapour generating device according to claim 1, wherein the locking mechanism comprises a bi-metallic portion.

3. The vapour generating device according to claim 1, further comprising a biasing mechanism arranged to bias the lid in an open position.

4. The vapour generating device according to claim 1, wherein the locking mechanism comprises a locking member arranged to retain the lid in the closed position.

5. The vapour generating device according to claim 4, wherein the locking member is moveable between a first position and a second position.

6. The vapour generating device according to claim 5, wherein the locking member is arranged to prevent the lid from moving from a closed position to an open position when the locking member is in the first position.

7. The vapour generating device according to claim 5, wherein the locking member is arranged to allow the lid to move from a closed position to an open position when the locking member is in the second position.

8. The vapour generating device according to claim 5, wherein the locking member is biased towards the first position.

9. The vapour generating device according to claim 1, wherein the locking mechanism comprises at least one intermediate part arranged to be connected to the bi-metallic portion of the locking mechanism.

10. The vapour generating device according to claim 9, wherein the at least one intermediate part is moveable between a third position and a fourth position.

11. The vapour generating device according to claim 10, wherein when the temperature of the heating chamber exceeds a predetermined threshold, the bi-metallic portion is arranged to have a shape such that the intermediate part is in the third position.

12. The vapour generating device according to claim 10, wherein when the temperature of the heating chamber is lower than a predetermined threshold, the bi-metallic portion is arranged to have a shape such that the intermediate part is in the fourth position.

13. The vapour generating device according to claim 5, wherein the locking member is arranged to be in the first position when the intermediate parts are in the third position.

14. The vapour generating device according to claim 10, wherein when the temperature of the heating chamber exceeds the predetermined threshold, the bi-metallic portion is arranged to move the intermediate part into the third position.

15. The vapour generating device according to claim 1, further comprising a heat insulator, the heat insulator arranged between the bi-metallic portion and an outer surface of the vapour generating device.
